(19) United States
(12) Patent Application Publication
(10) Pub. No.: US 2013/0131620 A1
(43) Pub. Date: May 23, 2013

(54) ABSORBENT ARTICLE

(71) Applicants: Takayuki Hisanaka, Kanonji-shi (JP); Nariaki Shimoe, Kanonji-shi (JP)

(72) Inventors: Takayuki Hisanaka, Kanonji-shi (JP); Nariaki Shimoe, Kanonji-shi (JP)

(73) Assignee: UNICHARM CORPORATION, Shikokuchuo-shi, Ehime-ken (JP)

(21) Appl. No.: 13/814,028

(22) PCT Filed: Nov. 21, 2011
(86) PCT No.: PCT/JP2011/006477

§ 371 Date: Feb. 4, 2013

(30) Foreign Application Priority Data
Nov. 26, 2010 (GB) 1020083.0

(51) Int. Cl. A61F 13/84

(52) U.S. Cl. CPC A61F 13/8405
USPC 604/367

(57) ABSTRACT

To demonstrate an effect of whitening easily-darkening wearer's groin portion, etc. An absorbent article according to the present invention comprises: a liquid permeable top sheet; a liquid impermeable back sheet; and an absorber disposed between the top sheet and the back sheet, wherein in both-side edges on a skin contacting surface side of the top sheet and along a longitudinal direction of the absorbent article, application regions to which a phosphoric ester type vitamin is applied are arranged intermittently.

### TECHNICAL FIELD

The present invention relates to an absorbent article.

### BACKGROUND ART

Patent Document 1 discloses an absorbent article, comprising: a liquid permeable top sheet; a liquid impermeable back sheet; an absorber disposed between the top sheet and the back sheet, wherein in order to protect the skin of a wearer, hydrophobic (i.e., lipophilic) esterified vitamin C is applied on a skin contacting surface side of the top sheet.

### CITATION LIST

### Patent Literature

[PTL 1] CN101507827A

### SUMMARY OF INVENTION

### Technical Problem

According to the above-described absorbent article, it is possible to demonstrate an effect of protecting the skin of the wearer, as described above. However, it is not possible to demonstrate an effect of whitening easily-darkened skin areas, for example, the wearer's groin portion, etc.

Therefore, the present invention has been achieved in view of the above-described problems, and an object thereof is to provide an absorbent article capable of demonstrating an effect of whitening easily-darkened skin areas.

A first characteristic of the present invention is an absorbent article which comprises: a liquid permeable top sheet; a liquid impermeable back sheet; an absorber disposed between the top sheet and the back sheet; and a pair of longitudinally extending leakage-preventing walls, one being provided along each side edge region of the absorbent article on the skin contacting surface side of the top sheet, wherein on both of the leakage preventing side walls a plurality of application regions to which a hydrophilic vitamin C derivative is applied are arranged, and the application regions are disposed intermittently.

Preferably, the application regions extend along the longitudinal direction of the absorbent article and are spaced from one another in a width direction of the absorbent article.

Preferably, the application regions extend continuously in the longitudinal direction.

Preferably, the application regions each extend parallel to a longitudinal center line of the absorbent article.

Preferably, the application regions comprise discrete shapes that are provided in a longitudinally extending pattern. The discrete shapes may comprise circles.

Preferably, the application regions are provided only on the leakage preventing walls.

Preferably, the vitamin C derivative comprises a phosphoric ester type vitamin C.

### BRIEF DESCRIPTION OF DRAWINGS

FIG. 1 is a plain view in which an absorbent article according to a first example, which does not constitute an embodiment of the present invention, is seen from a skin contacting surface side.

FIG. 2 is a plain view in which an absorbent article according to a first embodiment of the present invention is seen from a skin contacting surface side.

FIG. 3 is a partial plain view in which an absorbent article according to a modification is seen from a skin contacting surface side.

### DESCRIPTION OF EMBODIMENTS

### Example 1

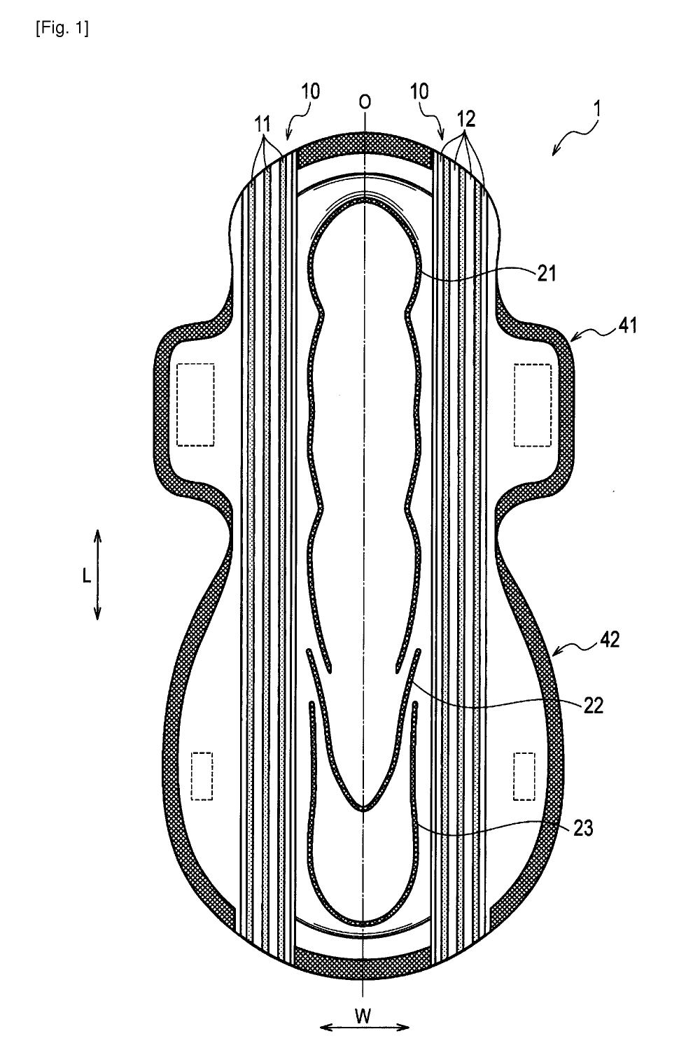

With reference to FIG. 1, an absorbent article 1 according to a first example not forming an embodiment of the present invention will be explained. For example, the absorbent article 1 according to this example is a panty liner, an incontinent pad, or a sanitary napkin, etc.

The absorbent article 1 according to this example includes: a liquid permeable top sheet, a liquid impermeable back sheet; and an absorber disposed between the top sheet and the back sheet. It is noted that in the absorbent article 1 according to this example, between the top sheet and the absorber, a second sheet may be disposed.

FIG. 1 shows a plain view in which the absorbent article according to the first example is seen from the skin contacting surface side.

The absorber is configured of crushed pulp, super absorbent polymer, and a core wrap wrapping the both. In this case, the super absorbent polymer is granular crosslinked polymer obtained by partially neutralizing polyacrylic acid with natrium.

Further, the core wrap is a sheet, made of an absorbent material. Examples of the core wrap material include tissue paper and an airlaid sheet that is obtained by converting a hydrophilic fiber or crushed pulp into a sheet by an airlaid method.

Moreover, in the absorbent article 1 according to this example, a plurality of compression grooves 21, 22, and 23 may be arranged, as shown in FIG. 1. In this case, the compression grooves 21, 22, and 23 are configured to compress various components ranging from the top sheet to the absorber.

Further, the absorbent article 1 according to this example may include a pair of wing portions 41 formed to protrude outwardly toward both sides in a width direction W of the absorbent article 1, as shown in FIG. 1. Moreover, the absorbent article 1 according to this example may include a pair of hip flap portions 42 behind the wing portions 41, in a longitudinal direction L of the absorbent article 1.

In this case, an adhesive for fixing the absorbent article 1 onto underwear is applied to a clothes contacting surface side of the back sheet, in the wing portions 41 and the hip flap portions 42.

Upon putting the absorbent article 1 on the underwear, a wearer wraps the wing portions 41 around a crotch of the underwear and develops the hip flap portions 42 over the underwear to fix the absorbent article 1 onto the underwear.

Moreover, as shown in FIG. 1, the absorbent article 1 according to this example includes: a plurality of application regions 11 to which hydrophilic vitamin C derivative is applied and a plurality of non-application regions 12 to which hydrophilic vitamin C derivative is not applied, at both-side edges 10 on the skin contacting surface side of the top sheet and along the longitudinal direction L of the absorbent article 1. The application regions 11 and the non-application regions 12 are disposed along the longitudinal direction L of the absorbent article 1 and disposed alternately in the width direction W of the absorbent article 1. Each application region 11 is lined in the width direction W and disposed intermittently. The application regions 11 extend along the longitudinal direction of the absorbent article and are spaced from one another in the width direction W of the absorbent article.

The vitamin C derivative is obtained by esterifying vitamin C with phosphoric acid in order to enhance permeability and absorbability to the skin, and to have an effect of whitening, of protecting the aging of the skin, or of removing depressed acne scarring as well.

The vitamin C derivative is more stable than vitamin C, and has a property that it is not easily oxidized, as well. Moreover, the vitamin C derivative changes itself to vitamin C in the skin with the help of a human enzyme (phosphatase) thereby to demonstrate its effect.

Vitamin C has an effect of inhibiting a pigment from depositing by blocking the production of melanin (improvement of freckle), an effect of reducing the produced melanin (lightening brown melanin), and a function of producing collagen.

The vitamin C, however, has a problem in that when it is ingested from a mouth through a food, etc., the ingested vitamin C is consumed at each body part after which the resultant amount is so small, thus not an sufficient amount to demonstrate a physiologic function for the health of the skin or surface texture.

This leads to a recent development of various products, such as a beauty product containing several % of vitamin C derivative, capable of directly absorbing vitamin C from the skin and demonstrating the effect of vitamin C for a long time at a location near a dermatological layer of the skin.

It is noted that the vitamin C derivative includes: “phosphoric ester type vitamin C”, which is a hydrophilic vitamin C derivative; and “next-generation vitamin C derivative of trisodium ascorbyl palmitate phosphate (APPS, Apprecier)”, which has both lipophilic and hydrophilic properties.

In this case, the phosphoric ester type vitamin C is also called “ascorbic acid phosphate vitamin C (VCP/AP)”. The phosphoric ester type vitamin C includes: “magnesium ascorbyl phosphate (VC-PMG, APM)”, “ascorbyl-3-phosphate sodium (AP-100)”, etc.

According to the absorbent article 1 according to this example, the application regions 11 to which the hydrophilic vitamin C derivative is applied are arranged on the skin contacting surface side of the top sheet and in the both-side edges 10 along the longitudinal direction L of the absorbent article 1, and thus, it is possible to demonstrate the effect of whitening the wearer's skin in the easily-darkened groin portion, etc.

Further, both the side edges 10 have the application regions 11 and the non-application regions 12, and thus, it is possible to demonstrate the effect of whitening the skin of the wearer in the application regions 11 while securing the liquid permeability in the non-application regions 12. In this example, the application regions 11 are disposed intermittently, and thus, if the balance between the application regions 11 and the non-application regions 12 is adjusted, then it becomes possible to maintain the balance between the whitening effect caused by the vitamin C derivative and the liquid permeability.

Moreover, generally, it is said that a speed at which the hydrophilic vitamin C derivative permeates through a skin is faster than that of the lipophilic vitamin C derivative. Therefore, by the provision of the hydrophilic vitamin C derivative, it becomes possible to demonstrate a rapid-acting whitening effect.

### First Embodiment

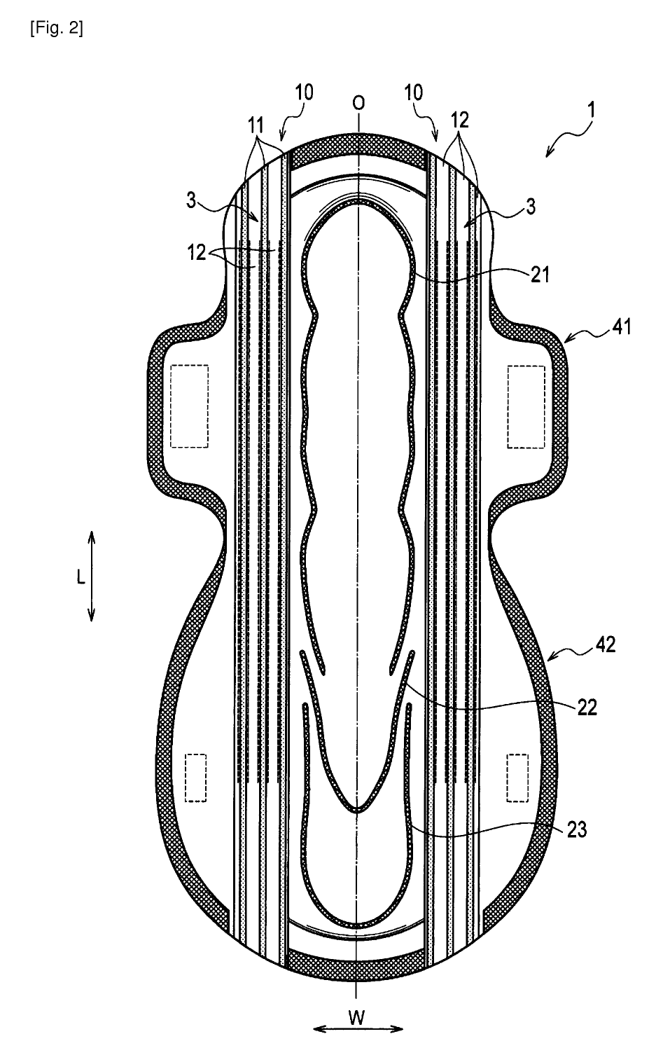

Hereinafter, with reference to FIG. 2, the absorbent article 1 according to a first embodiment, will be explained by a difference from the above-described absorbent article 1 according to the first example.

As shown in FIG. 2, in the absorbent article 1 according to the first embodiment, leakage-preventing walls 3 are arranged in both the side edges 10 along the longitudinal direction L of the absorbent article 1.

In the absorbent article 1 according to the first embodiment, the application regions 11 to which the hydrophilic vitamin C derivative is applied are arranged on the leakage-preventing walls 3 arranged in both the side edges 10.

(Modifications)

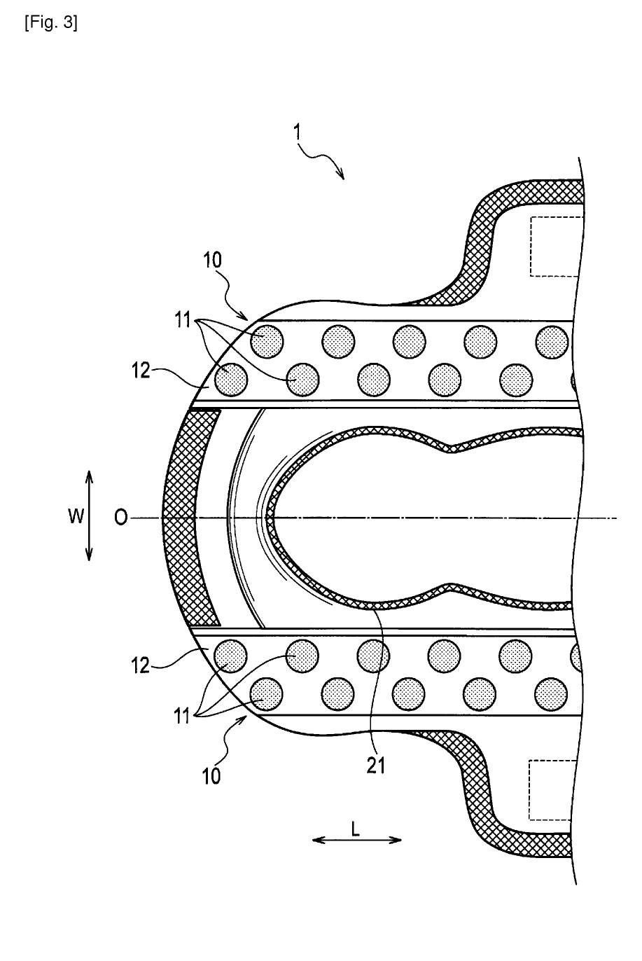

Hereinafter, with reference to FIG. 3, the absorbent article 1 according to a modification of the first example, a difference from the above-described absorbent article 1 according to the first example will be explained.

As shown in FIG. 3, the absorbent article 1 according to the modification includes a plurality of substantially circular application regions 11. To the application regions 11, the hydrophilic vitamin C derivative is applied, similarly to the absorbent article 1 according to the first example.

Further, in the absorbent article according to the present invention, for example, the same leakage-preventing walls 3 as those in the absorbent article according to the first embodiment are arranged in both the side edges 10 along the longitudinal direction L of the absorbent article 1 and provided with application regions 11 according to the first modification. Moreover, the application regions 11 of the absorbent article according to the modification, which are disposed along the longitudinal direction L, may also be disposed to be inclined relative to the longitudinal direction L. Further, one portion of the application regions 11 of the absorbent article according to the modification may be disposed in a crossing manner, and in the crossing portion, the application regions 11 may be disposed continuously. Moreover, the application regions 11 of the absorbent article according to the modification are circular with the same diameter; however, these regions 11 may be configured to be circular with a different diameter.

Thus, the present invention has been explained in detail by using the above-described embodiments; however, it is obvious that for persons skilled in the art, the present invention is not limited to the embodiments explained herein. The present invention can be implemented as a corrected, modified mode without departing from the scope of the present invention defined by the claims.

### INDUSTRIAL APPLICABILITY

As explained above, according to the present invention, it is possible to provide an absorbent article capable of demonstrating an effect of whitening easily-darkened skin areas.

### REFERENCE SIGNS LIST

1 absorbent article
10 both-side edges
11 application region
12 non-application region
21, 22, 23 compression groove
3 leakage-preventing wall
41 wing portion
42 hip flap portion

1. An absorbent article, comprising:
a liquid permeable top sheet;
a liquid impermeable back sheet;
an absorber disposed between the top sheet and the back sheet; and
a pair of longitudinally extending leakage-preventing walls, one being provided along each side edge region of the absorbent article on the skin contacting surface side of the top sheet,
wherein on both of the leakage preventing side walls a plurality of application regions to which a hydrophilic vitamin C derivative is applied are arranged, and the application regions are disposed intermittently.

2. The absorbent article according to claim 1, wherein the application regions extend along the longitudinal direction of the absorbent article and are spaced from one another in a width direction of the absorbent article.

3. The absorbent article according to claim 1, wherein the application regions extend continuously in the longitudinal direction.

4. The absorbent article according to claim 1, wherein the application regions each extend parallel to a longitudinal center line of the absorbent article.

5. The absorbent article according to claim 1, wherein the application regions comprise discrete shapes that are provided in a longitudinally extending pattern.

6. The absorbent article according to claim 5, wherein the discrete shapes comprise circles.

7. The absorbent article as claimed in claim 1, wherein the application regions are provided only on the leakage preventing walls.

8. The absorbent article as claimed in claim 1, wherein the vitamin C derivative comprises a phosphoric ester type vitamin C.
